(19) United States
(12) Patent Application Publication
(10) Pub. No.: US 2024/0157343 A1
(43) Pub. Date: May 16, 2024

(54) METHOD FOR THE HYDROGENATION OF AROMATIC NITRO COMPOUNDS

(71) Applicants: Covestro Deutschland AG, Leverkusen (DE); Lanxess Deutschland GmbH, Köln (DE)

(72) Inventors: Bernd Pennemann, Bergisch Gladbach (DE); Lennart Sandbrink, Langenfeld (DE); Eberhard Zirngiebl, Köln (DE); Thomas Kaese, Leverkusen (DE); Hans-Jürgen Quella, Leverkusen (DE); Paul Sprenger, Köln (DE); Daniel Ullrich, Monheim am Rhein (DE); Marc Williams, Köln (DE)

(21) Appl. No.: 18/546,817

(22) PCT Filed: Feb. 28, 2022
(86) PCT No.: PCT/EP2022/054929

§ 371 Date: Aug. 17, 2023

(30) Foreign Application Priority Data
Mar. 1, 2021 (EP) 21160014.3
Dec. 20, 2021 (EP) 21215937.0

(51) Int. Cl. B01J 23/80; B01J 21/08; B01J 23/745; B01J 23/75; B01J 23/847; B01J 23/889; B01J 35/40; B01J 35/51; B01J 35/55; B01J 37/02; B01J 37/08; C07C 209/36

(52) U.S. Cl. CPC B01J 23/80; B01J 21/08; B01J 23/745; B01J 23/75; B01J 23/8472; B01J 23/8892; B01J 35/40; B01J 35/51; B01J 35/55; B01J 37/0203; B01J 37/0205; B01J 37/0236; B01J 37/086; B01J 37/088; C07C 209/36

(57) ABSTRACT

The present invention relates (i) to a method for producing a doped copper-tetraammine-salt-based hydrogenation catalyst suitable for the hydrogenation of an aromatic nitro compound such that an aromatic amine is obtained, the hydrogenation catalyst comprising copper in metal form or in oxidic form and a doping metal selected from iron, cobalt, manganese, vanadium, zinc or a mixture of two or more thereof in metal form or in oxidic form on a carrier, the carrier comprising silicon dioxide shaped bodies and/or silicon carbide shaped bodies, (ii) to a doped copper-tetraammine-salt-based hydrogenation catalyst obtainable using the aforementioned method according to the invention, and (iii) to a method for producing an aromatic amine, comprising the hydrogenation of an aromatic nitro compound in the presence of a doped copper-tetraammine-salt-based hydrogenation catalyst comprising copper in metal form or in oxidic form and comprising a doping metal in metal form or in oxidic form on a carrier as hydrogenation catalyst, the carrier comprising silicon dioxide shaped bodies and/or silicon carbide shaped bodies, and the hydrogenation catalyst being, more particularly, the aforementioned hydrogenation catalyst according to the invention.

The present invention relates to (i) a process for preparing a hydrogenation catalyst, namely a process for preparing a doped tetraamminecopper salt-based hydrogenation catalyst suitable for hydrogenation of an aromatic nitro compound to obtain an aromatic amine, said hydrogenation catalyst comprising copper in metallic or oxidic form and a dopant metal selected from iron, cobalt, manganese, vanadium, zinc or a mixture of two or more of these in metallic or oxidic form on a support, where the support comprises shaped silicon dioxide bodies and/or shaped silicon carbide bodies, (ii) a hydrogenation catalyst, namely a doped tetraamminecopper salt-based hydrogenation catalyst obtainable by the aforementioned process of the invention, and (iii) a process for preparing an aromatic amine, said process comprising the hydrogenation of an aromatic nitro compound in the presence of a doped tetraamminecopper salt-based hydrogenation catalyst comprising copper in metallic or oxidic form and a dopant metal in metallic or oxidic form on a support as hydrogenation catalyst, where the support comprises shaped silicon dioxide bodies and/or shaped silicon carbide bodies, where the hydrogenation catalyst is especially the aforementioned hydrogenation catalyst of the invention.

The hydrogenation of nitroaromatics to the corresponding aromatic amines with hydrogen has long been known and is of major industrial significance. A representative example is the hydrogenation of nitrobenzene to aniline. The majority of the aniline produced globally is used for the production of the di- and polyamines of the diphenylmethane series (MDA), which in turn are intermediates for the production of the important di- and polyisocyanates of the diphenylmethane series (MDI).

The hydrogenation can be conducted in the liquid phase or gas phase, under isothermal or adiabatic conditions. Also known is a combination of isothermal and adiabatic reaction regimes. A series of catalysts has been described in the literature for this purpose. Particular mention should be made here of palladium- and copper-based catalyst systems.

For example, the use of palladium-based catalysts on ceramic supports is known. German patent application DE 28 49 002 A1 describes a process for reduction of nitro compounds in the presence of palladium-containing three-component supported catalysts in cooled tubular reactors. In preferred embodiments, the catalyst contains 1 to 20 g of palladium, 1 to 20 g of vanadium and 1 to 20 g of lead per liter of α-Al₂O₃. Similar catalysts, but additionally doped with Mo, Re or W, have also been described in DE 197 15 746 A1. EP 1 882 681 A1 discloses that it is advantageous to dope such three-component supported catalysts additionally with a sulfur- or phosphorus-containing, preferably phosphorus-containing, compound (for example the oxygen acids of phosphorus or the alkali metal salts thereof such as, in particular, sodium dihydrogenphosphate, sodium or potassium phosphate, or sodium hypophosphite). International publication WO 2013/030221 A1 describes the advantageous effects of potassium doping of the catalyst on the phenol content of the aniline formed.

The use of copper-based catalysts for the hydrogenation of nitrobenzene in particular has long been known (see U.S. Pat. Nos. 1,207,802 and 3,136,818). The support used for the catalytically active material was the natural stone pumice, which contains silicates and sodium as its main components.

The use of copper catalysts on silicon dioxide support for the hydrogenation of nitrobenzene to aniline has likewise long been known (e.g. GB 823,026 or U.S. Pat. No. 2,891,094 from the 1950s). Both patents describe the use of copper-ammine complexes as catalyst precursor compounds. For preparation of the catalysts, the hydrogel is precipitated by acidifying a sodium silicate solution, and it is admixed with the copper-ammine complex after filtration and washing. The hydrogel thus treated is filtered off, washed, dried, and calcined in reducing atmosphere. Although the treatment of the hydrogel with the copper-amine complex is described as impregnation, the procedure described, on account of the finely divided nature of the carrier (in the form of a freshly precipitated hydrogel and therefore not having any pores at all that could take up copper particles), is more of a simple deposition of copper particles on the hydrogel.

A tried-and-tested and frequently employed method of preparing hydrogenation catalysts is impregnation with metal salt solutions, in which the support used has pores that absorb the metal salt solutions. For this purpose, the support is either moistened with the metal salt solution up to a maximum of saturation of its water absorption capacity (called the “incipient wetness” method) or treated in supernatant solution. Impregnation methods are described, for example, in the following patent applications that are discussed hereinafter: WO 2010/130604 A2, EP 0 696 573 A1, DE 2 311 114, WO 95/32171 A1 and WO 2009/027135 A1.

International patent application WO 2010/130604 A2 describes a process for preparing aromatic amines, especially aniline, using copper-containing catalysts on SiO₂ supports. As well as copper, it is also possible to use further hydrogenation-active metals such as potassium (K), sodium (Na), barium (Ba), chromium (Cr), molybdenum (Mo), palladium (Pd), zinc (Zn), tungsten (W), nickel (Ni) or cobalt (Co). The application of the copper and such a further metal to the support is effected by joint impregnation. The process is more particularly characterized in that the SiO₂ has been produced by wet grinding, followed by spray drying. Wet grinding in the context of this patent application is understood to mean the comminuting of the already formed silicon dioxide (SiO₂) to particles of a particular size/diameter (page 6, third paragraph). According to this document, by wet grinding, it is possible to obtain SiO₂ particles of any desired size/diameter. All that are disclosed specifically, however, are silicon dioxide particles having a diameter in the order of magnitude of micrometers, preferably within a range from 1 to 35 m, especially 2 to 30 m (page 6, last paragraph). Example 1 discloses the production of a support with particle sizes within a range from 10 to 300 m (i.e. not more than 0.3 mm). Such small catalyst diameters are also required for the method described, since the catalysts thus produced are to be used in the form of fluidized bed catalysts, which would not even be practicable with macroscopically appreciable shaped bodies having sizes in the millimeter range. For application of the catalytically active metals, impregnation from supernatant solution as described, for example using ammoniacal carbonate solutions.

International patent application WO 2020/207874 A1 is also concerned with catalysts for hydrogenation of aromatic nitro compounds, the average particle sizes of which are at best in the region of tenths of millimeters (e.g. 114 m in example 2 and 118 m in example 3). What are described are catalyst systems that contain, as support, a component A, especially silicon carbide, and a component B1, especially silicon dioxide. Component A can be supplied to the reaction space, preferably a fluidized bed reactor, also separately from component B1 that has been provided with hydrogenation-active metals. A particular hydrogenation-active metal (B2) mentioned is copper. The catalyst may be doped with further metals (B3). These further metals B3 are preferably potassium (K), sodium (Na), barium (Ba), lead (Pb), zinc (Zn), vanadium (V), chromium (Cr), molybdenum (Mo), tungsten (W) or iron (Fe). The application of the metals B2 and B3 to the support B1 is effected by joint impregnation.

EP 0 696 573 A1 describes a process in which aromatic amines are prepared by hydrogenating the corresponding nitroaromatics in the gas phase over fixed bed catalysts. The catalysts contain hydrogenation-active metals on supports that can be prepared by impregnation. The hydrogenation catalyst used is especially a catalyst containing palladium on α-Al₂O₃, containing 1 to 100 g of Pd per liter of α-Al₂O₃, preferably precipitated in the form of a shell, where the catalyst may additionally contain vanadium and lead. There is no description of catalysts based on ammine complexes.

German published specification DE 2 311 114 is concerned with the improvement of copper chromite catalysts used for hydrogenation of ketones, carboxylic esters and nitro compounds. What is proposed for this purpose is a process for preparing a copper chromite catalyst applied to supports, which is more particularly characterized in that basic ammonium-copper(II) chromate is formed in the pores of an inorganic oxidic support material by reaction of precursors of basic ammonium-copper(II) chromate that react here with one another, and the support material is then heated to a temperature of about 250 to 500° C. for about 0.1 to 20 hours for conversion of the basic ammonium-copper(II) chromate to copper chromite. According to this document, copper chromite is often represented as “xCuO,Cr₂O₃”. It is immediately apparent to the person skilled in the art that this is a description of the stoichiometric ratios and does not give any information as to the actual structure of the catalyst.

International patent application WO 95/32171 A1 is concerned with the preparation of alcohols by the catalytic hydrogenation of the corresponding carbonyl compounds at elevated temperature and elevated pressure in the liquid phase. Copper catalysts are described for this purpose, these being obtainable by impregnating SiO₂-containing support materials with various copper salts that are thermally “readily” decomposable (i.e. below 350° C.), such as copper nitrate, copper carbonate, copper formate, copper oxalate and the readily water-soluble am(m)inic complexes thereof.

International patent application WO 2009/027135 A1 is likewise concerned with the preparation of alcohols by hydrogenation of carbonyl compounds. The hydrogenation catalyst used consists of a support material and at least one hydrogenation-active metal, wherein the support material is based on titanium dioxide, zirconium dioxide, aluminium oxide, silicon oxide or mixed oxides thereof, and the hydrogenation-active metal contains at least one element from the group of copper, cobalt, nickel, chromium, and wherein the support material further comprises the element barium. One example described is the preparation of a copper-containing impregnated catalyst on aluminium oxide with an about 14% tetraamminecopper carbonate solution.

German patent application DE 39 33 661 A1 is concerned with a catalyst for hydrogenation of acetophenone to methyl benzyl alcohol. The catalyst is prepared by impregnation, which is especially understood to mean spraying, of a silicon dioxide support with a solution of tetraamminecopper carbonate and a solution of ammonium chromate or mixture thereof, followed by drying. The spraying of a support with a metal salt solution is a method that can constitute an alternative to the above-described impregnation. Here too, the metal salt to be used may be used “in excess” (in accordance with the above-elucidated method of impregnation in supernatant solution) or may be matched to the absorption capacity of the pores of the support (in accordance with the above-described “incipient wetness” method).

German patent application DE 10 2010 029 924 A1 is concerned with the regeneration of copper-, chromium- and/or nickel-containing hydrogenation catalysts as used in the preparation of higher alcohols, especially those having 8 to 13 carbon atoms, by catalytic hydroformylation (also referred to as the oxo process) of the olefins having one carbon atom fewer, followed by hydrogenation of the aldehydes formed.

British patent GB 825,602 is concerned with the dehydrogenation of alcohols to aldehydes and ketones, using a catalyst containing reduced copper oxide and small amounts of non-reduced copper oxide, and also “alkali metal oxides”. The catalyst is prepared by heating a tetraamminecopper complex, followed by heating under nitrogen.

European patent application EP 3 320 969 A1 is concerned with chromium- and nickel-free catalysts for heterogeneous hydrogenation of oxo process aldehydes. The catalysts contain only copper, but it is necessary for the support material used to be silicon dioxide and for the content of Cu and SiO₂ in the active catalyst to be set accurately within very narrow limits.

In addition to use as catalysts for a wide variety of different reactions, copper compounds are also employed in many other fields, for example as a fungicide (see, for instance, U.S. Pat. No. 3,900,504).

As yet unpublished international patent application with application number PCT/EP2020/073991 describes a process for preparing an aromatic amine by hydrogenating an aromatic nitro compound, comprising the steps of (1) providing a tetraamminecopper salt-based impregnated catalyst, especially an impregnated catalyst obtainable by the incipient wetness method, comprising a metal or metal oxide on a support as hydrogenation catalyst, where at least metallic or oxidic copper (especially CuO) is present and the molar proportion of Cu based on all metals present is within a range from 0.75 to 1, and where the carrier comprises shaped silicon dioxide bodies or shaped silicon carbide bodies; (II) optionally activating the hydrogenation catalyst by treating with hydrogen in the absence of the aromatic nitro compound; (III) reacting the aromatic nitro compound with hydrogen in the presence of the optionally activated hydrogenation catalyst to obtain the aromatic amine. There is no disclosure of the use of dopant metals selected from iron, cobalt, manganese, vanadium, zinc or a mixture of two or more of these.

Entirely different from impregnated catalysts are catalyst alloys, for example the known Raney catalysts. WO 98/53910 A1 discloses a shaped activated fixed bed metal catalyst having a pore volume of 0.05 to 1 ml/g and an outer activated shell, consisting of a sintered, finely divided catalyst alloy and optionally promoters, wherein the catalyst alloy includes metallurgical phase domains that result from the preparation of the alloy, the greatest phase of which in terms of volume has a specific interfacial density of more than 0.5 μm⁻¹.

In addition to the use of palladium- or copper-based catalysts, the use of catalysts containing both metals is also known. One example of this is described in British patent GB 961,394. This describes catalysts for the treatment of motor vehicle exhaust gas, containing 0.5% to 25% copper and 0.01% to 3% palladium.

The prior art catalysts described for the hydrogenation of nitroaromatics, especially of nitrobenzene, are suitable in principle for this purpose, but there is still potential for improvement with regard to selectivity and long-term stability. The emphasis here was on the copper-based catalysts that are less expensive compared to the known palladium-based catalysts.

Taking account of the above, the present invention provides the following:

In a first aspect, the invention relates to a process for preparing a doped tetraamminecopper salt-based hydrogenation catalyst suitable for hydrogenation of an aromatic nitro compound to obtain an aromatic amine, said hydrogenation catalyst comprising copper in metallic or oxidic form and (at least) a dopant metal in metallic or oxidic form on a support, said support comprising shaped silicon dioxide bodies and/or (preferably or) shaped silicon carbide bodies (and especially not comprising any other support materials aside from silicon dioxide and/or silicon carbide), and said process comprising the steps of:
(a) dissolving a metal salt selected from an iron salt, a cobalt salt, a manganese salt, a vanadium salt, a zinc salt or a mixture of two or more of the aforementioned salts in water or aqueous ammonia solution to obtain an aqueous metal salt solution;
(b) treating the support with the aqueous metal salt solution to obtain a first impregnated catalyst precursor;
(c) drying the first impregnated catalyst precursor to obtain a first dried catalyst precursor;
(d) calcining the first dried catalyst precursor to obtain a first calcined catalyst precursor;
where steps (a) to (c) or (a) to (d) may also be conducted repeatedly (including with different metal salts);
(e) dissolving a copper salt in aqueous ammonia to obtain an ammoniacal copper salt solution;
(f) treating the first calcined catalyst precursor with the ammoniacal copper salt solution to obtain a second impregnated catalyst precursor;
(g) forming the doped tetraamminecopper salt-based hydrogenation catalyst by
   (1) drying the second impregnated catalyst precursor
   or
   (2) drying and calcining the second impregnated catalyst precursor,
where steps (e) to (g)(1) or (e) to (g)(2) may also be conducted repeatedly (including with different metal salts).

Entirely surprisingly, it has been found that a two-stage impregnation process leads to more active catalysts than a one-stage impregnation process, and the sequence of applying the metals to the support—first the dopant metal and then the copper—has a crucial influence on the quality of the catalyst.
In a second aspect, the invention relates to a doped tetraamminecopper salt-based hydrogenation catalyst obtainable by the aforementioned process of the invention.
In a third aspect, the invention relates to a process for preparing an aromatic amine by hydrogenating an aromatic nitro compound, comprising the steps of:
   (I) providing a doped tetraamminecopper salt-based hydrogenation catalyst according to the second aspect of the invention, i.e. providing a hydrogenation catalyst
      comprising copper in metallic or acidic form and (at least) a dopant metal in metallic or oxidic form on a support as hydrogenation catalyst,
      wherein the doped tetraamminecopper salt-based hydrogenation catalyst is obtainable by applying the dopant metal to the support, followed by applying the copper to the support containing the dopant metal,
      wherein the dopant metal is selected from iron, cobalt, manganese, vanadium, zinc or a mixture of two or more of these,
      and wherein the support comprises shaped silicon dioxide bodies and/or (preferably or) shaped silicon carbide bodies (and especially does not comprise any further support materials aside from silicon dioxide and/or silicon carbide);
   (II) optionally activating the hydrogenation catalyst by treating with hydrogen in the absence of the aromatic nitro compound;
   (III) reacting the aromatic nitro compound with hydrogen in the presence of the optionally activated hydrogenation catalyst to obtain the aromatic amine.

Forms of words such as “a dopant metal” or, referring thereto, “the dopant metal”, unless the context clearly suggests something different or something different is explicitly emphasized, do of course also include the case that two or more different dopant metals are used; such a course of action therefore does not leave the scope of the present invention. The situation is similar for forms of words such as “a metal”, “a metal oxide” and the like.

In the terminology of the present invention, a doped tetraamminecopper salt-based hydrogenation catalyst comprising a metal or metal oxide on a support is understood to mean a catalyst that has been obtained by treating, especially by impregnation or spraying, a support containing the dopant metal with an aqueous, especially ammoniacal, solution of a tetraamminecopper salt (i.e. a salt containing the tetraammine complex of Cu(II), namely the cation [Cu(NH₃)₄]²⁺), followed by drying and optionally calcining (preferably in oxygen-containing atmosphere).

The impregnation, the first preferred variant of the applying of the tetraammine copper salt to the support containing the dopant metal, is effected by mixing thereof with the aqueous, especially ammoniacal, solution of a tetraammine copper salt (by introducing the support containing the dopant metal into the tetraammine copper solution or by pouring the copper tetraammine solution over the support containing the dopant metal). The nature of the support containing the dopant metal and the amount of the aqueous, especially ammoniacal, solution of the tetraamminecopper salt are matched here to one another in such a way that
either (=impregnation in supernatant solution) there is more tetraammine salt solution present than the pores of the support containing the dopant metal (see also the section further down relating to shaped bodies) can accommodate,
or (=incipient wetness method—preferred method) there is a maximum of just as much (preferably somewhat less, especially only 95% to 99% or even only 96% to 98% of the maximum amount) tetraamminecopper solution as the pores of the support containing the dopant metal can accommodate.

The spraying, the second preferred variant of the applying of the tetraamminecopper salt to the support containing the dopant metal, is effected by spraying the support containing the dopant metal with an aqueous, especially ammoniacal, solution of the tetraamminecopper salt by means of one or more nozzles aligned into the rotating drum. In this method too, it is possible to use more tetraamminecopper salt solution than the pores of the support containing the dopant metal are able to accommodate (corresponding to impregnation in supernatant solution), or it is possible to use a maximum of just as much (preferably somewhat less, especially only 95% to 99% or even only 96% to 98% of the maximum amount) tetraamminecopper solution as the pores of the support containing the dopant metal are able to accommodate (corresponding to the incipient wetness method—here too the preferred method). The term incipient wetness method is also used hereinafter for spraying.

The hydrogenation catalyst is therefore especially one obtainable by the incipient wetness method mentioned (and preferably one that has indeed been produced by this method). In other words, this means that, in the process for production of the hydrogenation catalyst, the support containing the dopant metal is preferably treated with the aqueous, especially ammoniacal, solution of a tetraamminecopper salt so as not to exceed the maximum absorptivity of the support containing the dopant metal, as determined by saturation with water. Preferably, the amount of the copper salt solution is chosen such that it is within a range from 95% to 99%, more preferably within a range from 96% to 98%, of the maximum absorptivity. Means of determining the maximum absorptivity of the support containing the dopant metal, as determined by means of saturation with water are known in the specialist field. A crucial factor for the purposes of the present invention is the method described in the “Determination of the maximum absorptivity of the support” at the start of the examples section (this is applicable irrespective of whether the support material to be treated is an already doped support or a support that has not yet been doped with metals).

A support containing the dopant metal, in the terminology of the present invention, refers to a support that has been obtained by treatment, especially by impregnation or spraying, of the support with an aqueous solution of a salt of the dopant metal, followed by drying and calcination (preferably in oxygen-containing atmosphere). With regard to the use of the incipient wetness method described for the impregnation or spraying of copper, the same applies as to the impregnation of the starting support with dopant metal as well: Here too, preference is given to the employment of this method under the conditions described above.

According to the invention, the support comprises shaped silicon dioxide and/or silicon carbide bodies, a shaped body in this context being understood to mean that the shaped body is in the form of discrete (i.e. macroscopically appreciable) particles having average diameters especially within a range from 1.0 mm to 15 mm, preferably within a range from 4.0 mm to 15 mm, more preferably 4.0 to 10 mm. Examples especially include shaped cylindrical bodies and shaped spherical bodies, where the diameter of the footprint in the case of shaped cylindrical bodies is regarded as the diameter in this context and the length of the shaped cylindrical bodies is always greater than the diameter and is especially up to 2.0 times, preferably up to 1.8 times, more preferably up to 1.6 times, the diameter. In the case of shaped cylindrical bodies, the individual cylinders may also be combined to form aggregates comprising multiple cylinders, especially to give trilobes (aggregates composed of three cylinders joined to one another in longitudinal direction). In the case of such aggregates of cylinders, the diameter is considered to be the diameter of a theoretical circle encircling the bases of the mutually joined cylinders. Even in the case of such aggregates, the length of the cylindrical shaped bodies is always greater than the diameter and is especially up to 2.0 times, preferably up to 1.8 times, more preferably up to 1.6 times, the diameter.

The determination of an average diameter in the aforementioned sense is familiar to the person skilled in the art and can in principle be effected by all methods of determining particle sizes in the mm range that are known in the specialist field. In general, the result does not depend significantly on the type of method chosen. In the case of doubt (i.e. if, contrary to expectation, various methods of determining particle size that are acknowledged in the specialist field give significantly different results), the method described hereinafter is definitive for the purposes of the present invention:

The shaped bodies of the support that are to be analyzed are mixed, and then a representative sample of 20 shaped bodies is taken. A caliper gauge or a micrometer screw is then used to measure the above-defined diameter of each of the shaped bodies taken 3 times. The average is formed from the three individual measurements in each case, and the average is calculated in turn from the 20 averages thus obtained. This latter average refers to the average diameter in the context of the present invention. The measuring instrument used here is a caliper gauge when the average diameter is at least 4.0 mm. In the case of average diameters below 4.0 mm, a micrometer screw is used. In order to select the correct measuring instrument in a first step, it is generally sufficient to be guided by the manufacturer data for the support with regard to the particle sizes. If, however, a caliper gauge should be selected on the basis of such data, but an average diameter of less than 4.0 mm should be ascertained in the measurement (which may be the case in the case of manufacturer figures of or just slightly less than 4.0 mm), the measurement should be repeated with a micrometer screw. Suitable measuring instruments may be analog or digital and are available from specialist suppliers.

Shaped bodies as described are different than both unshaped structures (such as dust or hydrogels) and monolithic structures. The shaped silicon dioxide or silicon carbide bodies contain pores into which the aqueous solution of the tetraamminecopper salt penetrates. Silicon dioxide (SiO₂), as used in the terminology of the present invention, is typically referred to in the English-language literature as silica.

The molar proportion of Cu based on all metals present (=x(Cu)) is based in each case on the metals as such, i.e. x(Cu)=Molar amount Cu/Sum of the molar amounts of all metals present. The proportions by mass of the metals present on the catalyst are known from the preparation; the molar proportion of copper x(Cu) can easily be calculated therefrom.

What follows first is a brief summary of different possible embodiments of the invention, although the enumeration of embodiments should be considered to be nonexhaustive:

In a first embodiment of the process of the invention for production of a hydrogenation catalyst, which is combinable with all other embodiments, in step (b), for the treatment of 100 g of the support T, such a volume of aqueous metal salt solution V_{MS}(100 g T) is used that the ratio of the numerical value of the volume V_{MS}(100 g T) expressed in milliliters to the numerical value of the maximum absorptivity of the support S_{T} to be treated, expressed in percent, is not more than 1.00:

[V_{MS}(100 g T)/ml]/[S_{T}/%]≤1.00

where the maximum absorptivity of the support S_{T} is calculated from the ratio of the maximum mass of demineralized water m_{H2O} that can be absorbed by a sample P_{T} of the support to the mass of the sample of the support m_{PT}, multiplied by 100%:

S_{T}=[m_{H2O}/m_{PT}]×100%.

In a second embodiment of the process of the invention for production of a hydrogenation catalyst, which is a particular configuration of the first embodiment:

0.95≤[V_{MS}(100 g T)/ml]/[S_{T}/%]0.99.

In a third embodiment of the process of the invention for production of a hydrogenation catalyst, which is a further particular configuration of the first embodiment:

0.96≤[V_{MS}(100 g T)/ml]/[S_{T}/%]≤0.98.

In a fourth embodiment of the process of the invention for production of a hydrogenation catalyst, which is combinable with all other embodiments, in step (f), for the treatment of 100 g of the first calcined catalyst precursor KV1, such a volume of ammoniacal copper salt solution V_{KS}(100 g KV1) is used that the ratio of the numerical value of the volume V_{KS}(100 g KV1) expressed in milliliters to the numerical value of the maximum absorptivity of the first calcined catalyst precursor S_{KV1} to be treated, expressed in percent, is not more than 1.00:

[V_{KS}(100 g KV1)/ml]/[S_{KV1}/%]≤1.00

where the maximum absorptivity of the first calcined catalyst precursor S_{KV1} is calculated from the ratio of the maximum mass of demineralized water m_{H2O} that can be absorbed by a sample P_{KV1} of the first calcined catalyst precursor to the mass of the sample of the first calcined catalyst precursor m_{PKV1}, multiplied by 100%:

S_{KV1}=[m_{H2O}/m_{PKV1}]×100%.

In a fifth embodiment of the process of the invention for production of a hydrogenation catalyst, which is a particular configuration of the fourth embodiment:

0.95≤[V_{KS}(100 g KV1)/ml]/[S_{KV1}/%]≤0.99.

In a sixth embodiment of the process of the invention for production of a hydrogenation catalyst, which is a further particular configuration of the fourth embodiment:

0.96≤[V_{KS}(100 g KV1)/ml]/[S_{KV1}/%]≤0.98.

In a seventh embodiment of the process of the invention for production of a hydrogenation catalyst, which is combinable with all other embodiments, the metal salt comprises an iron salt, a zinc salt, a cobalt salt or a mixture of two or more of the aforementioned metal salts, and especially consists of one of the aforementioned.

In an eighth embodiment of the process of the invention for production of a hydrogenation catalyst, which is combinable with all other embodiments, the metal salt comprises a metal nitrate or metal oxalate, and is especially a metal nitrate or metal oxalate.

In a ninth embodiment of the process of the invention for production of a hydrogenation catalyst, which is combinable with all other embodiments, the metal salt comprises zinc(II) nitrate, iron(III) nitrate, cobalt(III) nitrate or a mixture of two or more of the aforementioned metal nitrates, and especially consists of one of the aforementioned. These of course also include hydrates (e.g. Zn(NO₃)₂·4H₂O) of the nitrates mentioned.

In a tenth embodiment of the process of the invention for production of a hydrogenation catalyst, which is combinable with all other embodiments, the dissolving in step (a) is conducted at temperatures within a range from 20° C. to 25° C.

In an eleventh embodiment of the process of the invention for production of a hydrogenation catalyst, which is combinable with all other embodiments, the drying in step (c) is conducted at temperatures within a range from 80° C. to 150° C., preferably 100° C. to 130° C.

In a twelfth embodiment of the process of the invention for production of a hydrogenation catalyst, which is combinable with all other embodiments, the calcining in step (d) is conducted at temperatures within a range from 300° C. to 600° C., preferably 400° C. to 500° C.

In a thirteenth embodiment of the process of the invention for production of a hydrogenation catalyst, which is combinable with all other embodiments, the copper salt comprises basic copper carbonate (copper hydroxide carbonate, e.g. malachite, CuCO₃·Cu(OH)₂; it is likewise possible to use other copper hydroxide carbonates having different ratios of “CuCO₃” to “Cu(OH)₂”).

In a fourteenth embodiment of the process of the invention for production of a hydrogenation catalyst, which is combinable with all other embodiments, in step (e), in addition to the copper salt, an ammonium salt, especially ammonium carbonate or ammonium acetate, is also dissolved in the aqueous ammonia.

In a fifteenth embodiment of the process of the invention for production of a hydrogenation catalyst, which is combinable with all other embodiments, the dissolving in step (e) is conducted at temperatures within a range from 0.0° C. to 25.0° C. (in the case of excess ammonia, corresponding to pH 9.0 or greater) or within a range from 0.0° C. to 10.0° C. (at lower pH).

In a sixteenth embodiment of the process of the invention for production of a hydrogenation catalyst, which is combinable with all other embodiments, the drying in step (g)(1) or step (g)(2) is conducted at temperatures within a range from 80° C. to 150° C.

In a seventeenth embodiment of the process of the invention for production of a hydrogenation catalyst, which is combinable with all other embodiments, the calcining in step (g)(2) is conducted at temperatures within a range from 300° C. to 600° C.

In an eighteenth embodiment of the process of the invention for production of a hydrogenation catalyst, which is combinable with all other embodiments, the ammoniacal copper salt solution has a pH (20° C.) within a range from 7.0 to 14.0, preferably 7.0 to 12.0.

In a nineteenth embodiment of the process of the invention for production of a hydrogenation catalyst, which is combinable with all other embodiments, a proportion by mass of copper compounds, calculated as metallic Cu, in the doped tetraamminecopper salt-based hydrogenation catalyst, based on the total mass thereof, is set within a range from 8% to 25%.

In a twentieth embodiment of the process of the invention for production of a hydrogenation catalyst, which is combinable with all other embodiments, a proportion by mass of metal compounds other than copper compounds, calculated as metals, in the doped tetraamminecopper salt-based hydrogenation catalyst, based on the total mass thereof, is set within a range from 0.1% to 25%, preferably 1.0% to 20%.

In a twenty-first embodiment of the process of the invention for production of a hydrogenation catalyst, which is combinable with all other embodiments, a molar proportion of Cu in the doped tetraamminecopper salt-based hydrogenation catalyst, based on all metals present, is set within a range from 0.30 to 0.99, preferably 0.45 to 0.95.

In a twenty-second embodiment of the process of the invention for production of a hydrogenation catalyst, which is combinable with all other embodiments, the treating in steps (b) and/or (f) comprises impregnating the support or the first catalyst precursor with the metal salt solution or the ammoniacal copper salt solution.

In a twenty-third embodiment of the process of the invention for production of a hydrogenation catalyst, which is combinable with all other embodiments, the treating in steps (b) and/or (f) comprises spraying the support or the first catalyst precursor with the metal salt solution or the ammoniacal copper salt solution.

In a twenty-fourth embodiment of the process of the invention for production of a hydrogenation catalyst, which is combinable with all other embodiments, the shaped silicon dioxide or silicon carbide bodies are (i) spheres, (ii) cylinders or (iii) aggregates of multiple cylinders joined to one another along their longitudinal axis and have an average diameter (determined with a caliper gauge or a micrometer screw by measuring 20 shaped bodies in each case and forming the average—see the above description of the method for details) within a range from 1.0 mm to 15 mm, preferably 4.0 mm to 15 mm, more preferably 4.0 mm to 10 mm, where the average diameter in the case of cylinders relates to the footprint of the cylinder, and in the case of aggregates composed of multiple cylinders joined to one another in their longitudinal direction to a circle that encloses the footprints of the mutually joined cylinders. In the case of cylindrical shaped bodies, irrespective of whether they are individual cylinders or aggregates of multiple cylinders, the length of the cylindrical shaped bodies is always greater than the diameter and is especially up to 2.0 times, preferably up to 1.8 times, more preferably up to 1.6 times, the diameter.

In a first embodiment of the process of the invention for production of an aromatic amine, which is combinable with all other embodiments, step (II) is conducted and the treatment with hydrogen is effected at temperatures in the range from 180° C. to 240° C.

In a second embodiment of the process of the invention for production of an aromatic amine, which is combinable with all other embodiments, step (III) is conducted
adiabatically at temperatures within a range from 160° C. to 500° C. or (preferably) 180° C. to 400° C.,
or
isothermally at temperatures in the range from 180° C. to 550° C. or (preferably) 190° C. to 400° C.

In a third embodiment of the process of the invention for production of an aromatic amine, which is combinable with all other embodiments, step (III) is conducted
adiabatically at a molar ratio of hydrogen to nitro groups within a range from 10 to 200,
or
isothermally at a molar ratio of hydrogen to nitro groups within a range from 3 to 100.

In a fourth embodiment of the process of the invention for production of an aromatic amine, which is combinable with all other embodiments, the proportion by mass of copper compounds, calculated as metallic Cu, in the hydrogenation catalyst provided in (1), based on the total mass thereof, is within a range from 8% to 25%.

In a fifth embodiment of the process of the invention for production of an aromatic amine, which is combinable with all other embodiments, the hydrogenation catalyst used is a tetraamminecopper carbonate-based hydrogenation catalyst.

In a sixth embodiment of the process of the invention for production of an aromatic amine, which is combinable with all other embodiments, the hydrogenation catalyst used is a tetraamminecopper carbonate ammonium salt-based hydrogenation catalyst, especially a tetraamminecopper carbonate ammonium carbonate-based hydrogenation catalyst or a tetraamminecopper carbonate ammonium acetate-based hydrogenation catalyst.

In a seventh embodiment of the process of the invention for production of an aromatic amine, which is combinable with all other embodiments, the proportion by mass of metal compounds other than copper compounds, calculated as metals, in the hydrogenation catalyst provided in (I), based on the total mass thereof, is set within a range from 0.1% to 25%, preferably 1.0% to 20%.

In an eighth embodiment of the process of the invention for production of a hydrogenation catalyst, which is combinable with all other embodiments, the dopant metal comprises iron, zinc, cobalt or a mixture of two or more of the aforementioned metals, and especially consists of one of the aforementioned.

In a ninth embodiment of the process of the invention for production of an aromatic amine, which is combinable with all other embodiments, the molar proportion of Cu in the doped tetraamminecopper salt-based hydrogenation catalyst, based on all metals present, is set within a range from 0.30 to 0.99, preferably 0.45 to 0.95.

In a tenth embodiment of the process of the invention for production of an aromatic amine, which is combinable with all other embodiments, step (I) comprises:
(a) dissolving a metal salt selected from an iron salt, a cobalt salt, a manganese salt, a vanadium salt, a zinc salt or a mixture of two or more of the aforementioned salts in water or aqueous ammonia solution to obtain an aqueous metal salt solution;
(b) treating the support with the aqueous metal salt solution to obtain a first impregnated catalyst precursor;
(c) drying the first impregnated catalyst precursor to obtain a first dried catalyst precursor;
(d) calcining the first dried catalyst precursor to obtain a first calcined catalyst precursor;
where steps (a) to (c) or (preferably) (a) to (d) may also be conducted repeatedly, especially twice (with the same or a different metal salt);
(e) dissolving a copper salt in aqueous ammonia to obtain an ammoniacal copper salt solution;
(f) treating the first calcined catalyst precursor with the ammoniacal copper salt solution to obtain a second impregnated catalyst precursor;
(g) forming the doped tetraamminecopper salt-based hydrogenation catalyst by
   (1) drying the second impregnated catalyst precursor
   or
   (2) drying and calcining the second impregnated catalyst precursor,
where steps (e) to (g)(1) or (preferably) (e) to (g)(2) may also be conducted repeatedly, especially twice (with the same or a different copper salt).

In an eleventh embodiment of the process of the invention for production of an aromatic amine, which is a particular configuration of the tenth embodiment, in step (b), for the treatment of 100 g of the support T, such a volume of aqueous metal salt solution V_{MS}(100 g T) is used that the ratio of the numerical value of the volume V_{MS}(100 g T) expressed in milliliters to the numerical value of the maximum absorptivity of the support S_{T} to be treated, expressed in percent, is not more than 1.00:

[V_{MS}(100 g T)/ml]/[S_{T}/%]≤1.00

where the maximum absorptivity of the support S_{T} is calculated from the ratio of the maximum mass of demineralized water m_{H2O} that can be absorbed by a sample P_{T} of the support to the mass of the sample of the support m_{PT}, multiplied by 100%:

S_{T}=[m_{H2O}/m_{PT}]×100%.

In a twelfth embodiment of the process of the invention for production of an aromatic amine, which is a particular configuration of the eleventh embodiment:

0.95≤[V_{MS}(100 g T)/ml]/[S_{T}/%]≤0.99.

In a thirteenth embodiment of the process of the invention for production of an aromatic amine, which is a particular configuration of the eleventh embodiment:

0.96≤[V_{MS}(100 g T)/ml]/[S_{T}/%]≤0.98.

In a fourteenth embodiment of the process of the invention for production of an aromatic amine, which is a particular configuration of the tenth to thirteenth embodiments, in step (f), for the treatment of 100 g of the first calcined catalyst precursor KV1, such a volume of ammoniacal copper salt solution V_{KS}(100 g KV1) is used that the ratio of the numerical value of the volume V_{KS}(100 g T) expressed in milliliters to the numerical value of the maximum absorptivity of the first calcined catalyst precursor S_{KV1} to be treated, expressed in percent, is not more than 1.00:

[V_{KS}(100 g KV1)/ml]/[S_{KV1}/%]≤1.00

where the maximum absorptivity of the first calcined catalyst precursor S_{KV1} is calculated from the ratio of the maximum mass of demineralized water m_{H2O} that can be absorbed by a sample P_{KV1} of the first calcined catalyst precursor to the mass of the sample of the first calcined catalyst precursor m_{PKV1}, multiplied by 100%:

S_{KV1}=[m_{H2O}/m_{PKV1}]×100%.

In a fifteenth embodiment of the process of the invention for production of an aromatic amine, which is a particular configuration of the fourteenth embodiment:

0.95≤[V_{KS}(100 g KV1)/ml]/[S_{KV1}/%]≤0.99.

In a sixteenth embodiment of the process of the invention for production of an aromatic amine, which is a further particular configuration of the fourteenth embodiment:

0.96≤[V_{KS}(100 g KV1)/ml]/[S_{KV1}/%]≤0.98.

In a seventeenth embodiment of the process of the invention for production of an aromatic amine, which is a particular configuration of the fourteenth to sixteenth embodiments, the metal salt comprises a metal nitrate or metal oxalate.

In an eighteenth embodiment of the process of the invention for production of an aromatic amine, which is a particular configuration of the tenth to seventeenth embodiments, the metal salt comprises zinc(II) nitrate, iron(III) nitrate, cobalt(III) nitrate or a mixture of two or more of the aforementioned metal nitrates comprises, and especially consists of one of the aforementioned.

In a nineteenth embodiment of the process of the invention for production of an aromatic amine, which is a particular configuration of the tenth to eighteenth embodiments, the dissolving in step (I)(a) is conducted at temperatures within a range from 20.0° C. to 25° C.

In a twentieth embodiment of the process of the invention for production of an aromatic amine, which is a particular configuration of the tenth to nineteenth embodiments, the drying in step (I)(c) is conducted at temperatures within a range from 80° C. to 150° C.

In a twenty-first embodiment of the process of the invention for production of a hydrogenation catalyst, which is a particular configuration of the tenth to twentieth embodiments, the calcining in step (I)(d) is conducted at temperatures within a range from 300° C. to 600° C.

In a twenty-second embodiment of the process of the invention for production of a hydrogenation catalyst, which is a particular configuration of the tenth to twenty-first embodiments, the copper salt comprises basic copper carbonate (copper hydroxide carbonate, CuCO₃·Cu(OH)₂).

In a twenty-third embodiment of the process of the invention for production of an aromatic amine, which is a particular configuration of the tenth to twenty-second embodiments, in step (I)(e), in addition to the copper salt, an ammonium salt, especially ammonium carbonate or ammonium acetate, is also dissolved in the aqueous ammonia.

In a twenty-fourth embodiment of the process of the invention for production of an aromatic amine, which is a particular configuration of the tenth to twenty-third embodiments, the dissolving in step (I)(e) is conducted at temperatures within a range from 0.0° C. to 25.0° C. (in the case of excess ammonia, corresponding to pH 9.0 or greater) or within a range from 0.0° C. to 10.0° C. (at lower pH).

In a twenty-fifth embodiment of the process of the invention for production of an aromatic amine, which is a particular configuration of the tenth to twenty-fourth embodiments, the drying in step (I)(g)(1) or step (I)(g)(2) is conducted at temperatures within a range from 80° C. to 150° C.

In a twenty-sixth embodiment of the process of the invention for production of an aromatic amine, which is a particular configuration of the tenth to twenty-fifth embodiments, the calcining in step (I)(g)(2) is conducted at temperatures within a range from 300° C. to 600° C.

In an twenty-seventh embodiment of the process of the invention for production of an aromatic amine, which is a particular configuration of the tenth to twenty-sixth embodiments, the ammoniacal copper salt solution has a pH (20° C.) within a range from 7.0 to 14, preferably 7.0 to 12.0.

In an twenty-eighth embodiment of the process of the invention for production of an aromatic amine, which is a particular configuration of the tenth to twenty-seventh embodiments, the treating in steps (I)(b) and/or (I)(f) comprises impregnating the support or the first catalyst precursor with the metal salt solution or the ammoniacal copper salt solution.

In a twenty-ninth embodiment of the process of the invention for production of an aromatic amine, which is a particular configuration of the tenth to twenty-seventh embodiments, the treating in steps (I)(b) and/or (I)(f) comprises spraying the support or the first catalyst precursor with the metal salt solution or the ammoniacal copper salt solution.

In a thirtieth embodiment of the process of the invention for production of an aromatic amine, which is combinable with all other embodiments, the optionally activated hydrogenation catalyst is arranged in a fixed catalyst bed in step (III).

In a thirty-first embodiment of the process of the invention for production of an aromatic amine, which is combinable with all other embodiments, an aromatic nitro compound of the formula

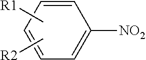

is hydrogenated, in which R1 and R2 are independently hydrogen, methyl or ethyl, where R2 may additionally also be NO₂.

The embodiments briefly outlined above and further possible configurations of the invention are more particularly elucidated hereinafter. In this context, all embodiments and further configurations of the invention are combinable as desired with one another unless the opposite is clearly apparent to a person skilled in the art from the context or any different statement is made explicitly.

Provision of the Catalyst for Performance of the Hydrogenation

The production of the hydrogenation catalyst in the first aspect of the invention comprises the following steps:
(a) dissolving a metal salt selected from an iron salt, a cobalt salt, a manganese salt, a vanadium salt, a zinc salt or a mixture of two or more of the aforementioned salts in water or aqueous ammonia solution to obtain an aqueous metal salt solution;
(b) treating the support with the aqueous metal salt solution to obtain a first impregnated catalyst precursor;
(c) drying the first impregnated catalyst precursor to obtain a first dried catalyst precursor;
(d) calcining the first dried catalyst precursor to obtain a first calcined catalyst precursor;
where steps (a) to (c) or (preferably) (a) to (d) may also be conducted repeatedly, especially twice (with the same or a different metal salt);
(e) dissolving a copper salt in aqueous ammonia to obtain an ammoniacal copper salt solution;
(f) treating the first calcined catalyst precursor with the ammoniacal copper salt solution to obtain a second impregnated catalyst precursor;
(g) forming the doped tetraamminecopper salt-based hydrogenation catalyst by
   (1) drying the second impregnated catalyst precursor
   or
   (2) drying and calcining the second impregnated catalyst precursor,
where steps (e) to (g)(1) or (preferably) (e) to (g)(2) may also be conducted repeatedly, especially twice (with the same or a different copper salt).

The treatment of the support or the first catalyst precursor with a solution of the dopant metal or copper salt can be effected by impregnating or spraying, as already elucidated further up. Treatment by impregnating is preferred. Both techniques are well known in the specialist field and therefore need not be elucidated in detail here.

Both in the case of employment of impregnation and in the case of employment of spraying, it is preferable to use the solution of the dopant metal or copper salt in such a ratio to the support (T) or to the first calcined catalyst precursor (KV1) as not to exceed the maximum absorptivity S of the support (S_{T}) or of the first calcined catalyst precursor (S_{KV1}) (incipient wetness method), where the maximum absorptivity of the support or of the first catalyst precursor is calculated from the ratio of the maximum mass of demineralized water m_{H2O} that can be absorbed by a sample of the support or of the first calcined catalyst precursor to the mass of the sample of the support (m_{PT}) or of the first calcined catalyst precursor (m_{PKV1}), multiplied by 100%:

S_{T}=[m_{H2O}/m_{PT}]×100%.

S_{KV1}=[m_{H2O}/m_{PKV1}]×100%.

What is meant in the context of the present invention by not exceeding the maximum absorptivity S is that, for the treatment of 100 g of the support T or of 100 g of the first calcined catalyst precursor KV1, such a volume of aqueous metal salt solution V_{MS}(100 g T) or copper salt solution V_{KS}(100 g KV1) is used that the ratio of the numerical value of the volume the metal salt solution V_{MS}(100 g T) or of the copper salt solution V_{KS}(100 g T), expressed in milliliters, to the numerical value of the maximum absorptivity of the support S_{T} to be treated or of the first calcined catalyst precursor S_{KV1} to be treated, expressed in percent, is not more than 1.00.

The following condition therefore preferably applies:

[V_{MS}(100 g T)/ml]/[S_{T}/%]≤1.00.

More preferably, 0.95≤[V_{MS}(100 g T)/ml]/[S_{T}/%]≤0.99; most preferably, 0.96≤[V_{MS}(100 g T)/ml]/[S_{T}/%]≤0.98.

It is additionally preferable that:

[V_{KS}(100 g KV1)/ml]/[S_{KV1}/%]≤1.00.

More preferably, 0.95≤[V_{KS}(100 g KV1)/ml]/[S_{KV1}/%]≤0.99; most preferably, 0.96≤[V_{KS}(100 g KV1)/ml]/[S_{KV1}/%]≤0.98.

Irrespective of the manner of treatment and the choice of ratios in the treatment step, it is preferable to use, as metal salt, an iron salt, a zinc salt, a cobalt salt or a mixture of two or more of the aforementioned metal salts. Particular preference is given to using metal nitrates or metal oxalates. The metal salt is most preferably zinc(II) nitrate, iron(II) nitrate, cobalt(III) nitrate or a mixture of two or more of the aforementioned metal nitrates.

The dissolving of the metal salts in step (a) is not temperature-critical and can be conducted at ambient temperature, e.g. 20° C. to 25° C. In the treatment of the support with the metal salt solution in step (b), it is possible to leave the moist support to stand for a prolonged period of time before the next step is conducted (“aging”). However, this is not essential.

Useful temperatures for the drying step (c) have been found to be within a range from 80° C. to 150° C. The calcination in step (d) is preferably conducted at temperatures within a range from 300° C. to 600° C. Drying and calcination are preferably conducted in an oxygenous atmosphere, especially air. But calcination in an inert gas atmosphere (for example in a nitrogen atmosphere) is likewise conceivable.

The copper salt used is preferably basic copper carbonate (copper hydroxide carbonate, CuCO₃·Cu(OH)₂). Irrespective of the exact type of copper salt, it has been found to be useful, in step (e), in addition to the copper salt, also to dissolve an ammonium salt, such as ammonium carbonate or ammonium acetate in particular, in the aqueous ammonia. Step (e) is preferably conducted at temperatures within a range from 0.0° C. to 25.0° C. (especially in the case of excess ammonia, corresponding to pH 9.0 or greater) or within a range from 0.0° C. to 10.0° C. (especially at lower pH). It is preferable that the ammoniacal copper salt solution has a pH (20° C.) within a range from 7.0 to 14.0, preferably 7.0 to 12.0. In the treatment of the first calcined catalyst precursor with the copper salt solution in step (f), it is possible to leave the moist first calcined catalyst precursor to stand for a prolonged period of time before the next step is conducted (“aging”). However, this is not essential.

The drying in step (g)(1) or step (g)(2) is preferably conducted at temperatures within a range from 80° C. to 150° C. If a calcination is conducted as per step (g)(2), this is preferably effected at temperatures within a range from 300° C. to 600° C. Drying and calcination are preferably conducted in an oxygenous atmosphere, especially air. But calcination in an inert gas atmosphere (for example in a nitrogen atmosphere) is likewise conceivable.

The process of the invention is preferably conducted so as to establish a proportion by mass of copper compounds, calculated as metallic Cu, in the doped tetraamminecopper salt-based hydrogenation catalyst, based on the total mass thereof, within a range from 8% to 25%. Preference is given to establishing a proportion by mass of metal compounds other than copper compounds, calculated as metals, in the doped tetraamminecopper salt-based hydrogenation catalyst, based on the total mass thereof, within a range from 0.1% to 25%, preferably 1.0% to 20%. Preferably, the molar proportion of Cu based on all metals present in the doped tetraamminecopper salt-based hydrogenation catalyst is adjusted to a value within a range from 0.30 to 0.99, preferably 0.45 to 0.95. Accordingly, the molar proportion of all dopant metals based on all the metals present is preferably from 0.01 to 0.70, preferably 0.05 to 0.55.

By employing the above-described process for producing a hydrogenation catalyst, the hydrogenation catalyst according to the second aspect of the invention is obtainable.

Hydrogenation Procedure

The process for producing an aromatic amine by hydrogenating an aromatic nitro compound in the third aspect of the invention comprises the following steps:
(I) providing a doped tetraamminecopper salt-based hydrogenation catalyst according to the second aspect of the invention, i.e. providing a hydrogenation catalyst
   comprising copper in metallic or acidic form and (at least) a dopant metal in metallic or oxidic form on a support as hydrogenation catalyst,
   wherein the copper catalyst is obtainable by applying the dopant metal to the support, followed by applying the copper to the support containing the dopant metal, and is especially obtainable by the process of the invention for producing a hydrogenation catalyst (i.e. the hydrogenation catalyst is especially the hydrogenation catalyst of the invention),
   wherein the dopant metal is selected from iron, cobalt, manganese, vanadium, zinc or a mixture of two or more of these,
   and wherein the support comprises shaped silicon dioxide bodies and/or shaped silicon carbide bodies;
(II) optionally activating the hydrogenation catalyst by treating with hydrogen in the absence of the aromatic nitro compound;
(III) reacting the aromatic nitro compound with hydrogen in the presence of the optionally activated hydrogenation catalyst to obtain the aromatic amine.

Preferably, the proportion by mass of copper compounds, calculated as metallic Cu, in the hydrogenation catalyst provided in (I), based on the total mass thereof, is in the range from 8% to 25%. The proportion by mass of metal compounds (calculated as metal) other than copper compounds in the hydrogenation catalyst provided in step (I), based on the total mass thereof, is preferably within a range from 0.1% to 25%, preferably 1.0% to 20%. It is preferable that the molar proportion of Cu based on all metals present in the doped tetraamminecopper salt-based hydrogenation catalyst is within a range from 0.30 to 0.99, preferably 0.45 to 0.95. Accordingly, the molar proportion of all metals other than copper is preferably from 0.01 to 0.70, preferably 0.05 to 0.55. The dopant metal is especially iron, zinc, cobalt or a mixture of two or more of the aforementioned metals.

The hydrogenation catalyst is especially a tetraamminecopper carbonate-based hydrogenation catalyst. The hydrogenation catalyst used is more preferably a tetraamminecopper carbonate ammonium salt-based hydrogenation catalyst, especially a tetraamminecopper carbonate ammonium carbonate-based hydrogenation catalyst or a tetraamminecopper carbonate ammonium acetate-based hydrogenation catalyst.

For further details of the hydrogenation catalyst to be provided in step (I), reference may be made to the description of the process for producing a hydrogenation catalyst in the first aspect of the invention.

It is preferable to conduct the activation in step (II). Useful temperatures for this step have been found to be within a range from 180° C. to 240° C.

The actual hydrogenation, step (III), can be conducted adiabatically (i.e. without cooling or heating) or isothermally (i.e. with cooling by removing the heat of reaction, for example by using a shell and tube reactor with a heat carrier that flows around the tubes). In the case of an adiabatic reaction regime, useful molar ratios of hydrogen to nitro groups have been found to be within a range from 10 to 200, and useful temperatures within a range from 160° C. to 500° C. or (preferably) 180° C. to 400° C. In the case of an isothermal reaction regime, useful molar ratios of hydrogen to nitro groups have been found to be within a range from 3 to 100, and useful temperatures within a range from 180° C. to 550° C. or (preferably) 190° C. to 400° C.

It is preferable to arrange the hydrogenation catalyst for performance of step (III) in a fixed catalyst bed. The hydrogenation process of the invention has been found to be particularly useful for hydrogenation of aromatic nitro compounds of the following formula:

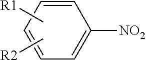

in which R1 and R2 are independently hydrogen, methyl or ethyl, where R2 may additionally also be NO₂.

The invention is elucidated in detail hereinafter with the aid of examples.

### EXAMPLES

General Methods

Determination of Maximum Absorptivity of the Support

The absorptivity maximum is determined by weighing the shaped bodies before and after absorption of water, as described hereinafter. For this purpose, the support material TM (either untreated support or support that has already been treated with dopant metal or copper) is weighed out and left to stand under demineralized water (DM water) until no further air bubbles ascend. The supernatant water is decanted, and the outside of the moist shaped bodies is dried by rolling on filter paper. By weighing the shaped bodies that have been dried on the outside in this way and subtracting the starting weight, the water absorption in grams is obtained, corresponding to the absorptivity maximum of the shaped body used. In the case of repeated impregnation, the determination of the absorptivity of the support material is conducted before each impregnation. The maximum absorptivity S, in the terminology of the present invention, is expressed as a percentage:

S=[(mass of water absorbed)/(mass of the support material before the absorption of water)]×100%.

For the purpose of calculating the amount of impregnating salt solution to be used, the volume of the metal salt solution V_{MS} or copper salt solution V_{KS} to be used for the impregnation, the numerical value of which in ml corresponds to the numerical value of maximum absorptivity S in %, is set as that impregnation volume which is the maximum that can be absorbed by 100 g of the support material used.

In the examples that follow, for the impregnation of 100 g of the support material, an impregnation volume of metal salt solution V_{MS}(100 g TM) or copper salt solution V_{KS}(100 g TM) that corresponded to 98% of the value thus ascertained for S was used. This applies to all impregnation steps (i.e. both to the application of the dopant metal and to the application of the copper and, in the case of multiple impregnations, to each individual impregnation step).

### Calculation Examples

a) The absorptivity of “SS69138” shaped SiO₂ bodies (3 mm extrudates) from Saint-Gobain Norpro (used as support in all experiments) was found to be 108.5%. For impregnation of 100 g of these shaped bodies up to the maximum absorptivity S in the terminology of the present invention, 108.5 ml of impregnation salt solution was accordingly required. The amount used was in fact [108.5 ml×0.98=] 106.3 ml of impregnation solution per 100 g of shaped bodies.
b) The absorptivity of Zn-doped shaped SiO₂ bodies was found to be 88.8%. For the copper impregnation of 100 g of the Zn-doped shaped SiO₂ bodies, therefore, [88.8 ml×0.98=]87.0 ml of tetraamminecopper solution was used.

General Procedure for Production of the Catalysts

21 catalysts were produced. Details can be found in table 1. Unless explicitly stated otherwise in table 1, the following conditions were observed:

1. Impregnation of Support with Dopant Metal

Production of the Impregnation Solution (Step (a)):

The amount of metal salt required is dissolved in 75 ml of demineralized water (DM water) and made up to the required volume of impregnation solution with DM water.

Performance of the Impregnation (Step (b)):

100 g of the SiO₂ support is added to the aqueous metal salt solution while mixing with tumbling movements. After 10 minutes in motion, the absorption of liquid is complete.

Performance of the Drying (Step (c)):

The impregnated support is dried in a hot air drier at 120° C. for 40 min.

Performance of the Calcination (Step (d)):

The dried support that has been impregnated with the dopant metal is heated up to 450° C. in a static oven with a ramp of 3° C./min, and this temperature is maintained for 4 h. After cooling to room temperature, steps (a) to (d) may be repeated.

2. Impregnation of the Doped Support with Copper

Production of the Tetraamminecopper Salt Solution (Step (e)):

Masses used for a solution with a proportion by mass of Cu of (10.0±0.5)% at pH=10.0±1.0:
ammonium carbonate 316 g
basic copper carbonate 364 g
25-30% ammonia 652 g
demineralized water 668 g

First of all, the starting materials are cooled to below 5° C. in a refrigerator. Water and ammonia are mixed in a closable vessel. The solids are weighed out together in a dish and added rapidly to the cooled ammonia solution, and they are mixed together with the lid closed (with a pressure-equalizing valve for safety reasons) until the salts have dissolved. A dark blue solution having a copper concentration of 10% by mass and a pH of 10 was obtained. The density of the solution at room temperature was found to be 1.22 g/ml.

Performance of the Impregnation (Step (f)):

The support material from step 1 that has been doped with one or more dopant metals is added to the required proportion of the tetraamminecopper salt solution while mixing with tumbling movements. After 10 minutes in motion, the absorption of liquid had ended.

Performance of the Drying (Step (g) (1)):

The support material impregnated with tetraamminecopper salt solution is dried in a hot air drier at 120° C. for 60 min. A color change from dark blue to green or black is observed here.

Performance of the Calcination (Step (g) (2), Optional):

The dried support material is heated up to 450° C. with a ramp of 3° C./min and kept at that temperature for 4 h. The resultant black catalyst particles are cooled down to room temperature within about 8 h hours. After cooling to room temperature, steps (e) to (g)(1) or (e) to (g)(2) may be repeated.

TABLE 1
Overview of the catalyst preparations conducted

| | | | | Max. absorptivity S/% | | | |
|---|---|---|---|---|---|---|---|
| | | | Type of hydrogenation | Before | Before | Before | |
| | | | catalyst/optionally | 1st | 2nd | 3rd | |
| Ex. | Brief | Type of | sequence of | impreg- | impreg- | impreg- | Dopant |
| no. | description | example | drying | nation | nation | nation | metal 1 |
| K1 | Cu only | comp. | Tetraamminecopper | 119.0 | n.a. | n.a. | n.a. |
| K2 | Dopant metal | inv. | Zinc nitrate, then | 108.0 | 97.5 | n.a. | 4.6% Zn |
| | and copper. | | tetraamminecopper | | | | |
| K3 | impregnated | comp. | Tetraamminecopper. | 108.0 | 89.0 | n.a. | 4.6% Zn |
| | 1x each | | then zinc nitrate | | | | |
| K4 | | comp. | Tetraamminezinc and -copper | 108.0 | n.a. | n.a. | 3.5% Zn |
| | | | (impregnated together) | | | | |
| K5 | | inv. | Iron nitrate. then | 107.5 | 104.0 | n.a. | 1.8% Fe |
| | | | tetraamminecopper | | | | |
| K6 | | inv. | Cobalt nitrate. then | 107.5 | 107.0 | n.a. | 1.8% Co |
| | | | tetreamminecopper | | | | |
| K7 | | inv. | Manganese nitrate, | 107.5 | 106.0 | n.a. | 1.8% Mn |
| | | | then tetraamminecopper | | | | |
| K8 | | comp. | Magnesium nitrate, | 107.5 | 103.0 | n.a. | 1.8% Mg |
| | | | then tetraamminecopper | | | | |
| K9 | | inv. | Vanadium oxalate, | 107.5 | 103.0 | n.a. | 1.8% V |
| | | | then tetraamminecopper | | | | |
| K10 | Cu only | comp. | Copper nitrate | 108.0 | n.a. | n.a. | n.a. |
| K11 | | comp. | 2 x tetraamminecopper | 119.0 | 92.0 | n.a. | n.a. |
| K12 | Dopant metal | inv. | Zinc nitrate, then 2x | 108.5 | 89.0 | 77.5 | 7.9% Zn |
| | impregnated | | tetraamminecopper | | | | |
| K13 | 1x, copper 2x | comp. | Zinc nitrate, then | 108.5 | 84.5 | 61.0 | 9.9% Zn |
| | | | 2x copper nitrate | | | | |
| K14 | | inv. | Iron nitrate, | 107.5 | 106.0 | 83.5 | 1.6% Fe |
| | | | then 2x | | | | |
| | | | tetraamminecopper | | | | |
| K15 | 2 dopant | inv. | Iron nitrate, then | 107.5 | 103.0 | 96.0 | 1.8% Fe |
| | metals, copper | | zinc nitrate, then | | | | |
| | impregnated 1x | | tetraamminecopper | | | | |
| K16 | | inv. | Zinc nitrate, then | 107.5 | 99.0 | 96.0 | 4.5% Zn |
| | | | iron nitrate. then | | | | |
| | | | tetraamminecopper | | | | |
| K17 | | inv. | Mixture of iron nitrate | 107.6 | 96.0 | n.a. | 1.8% Fe |
| | | | and zinc nitrate, then | | | | |
| | | | tetraamminecopper | | | | |
| K18 | | inv. | Zinc nitrate, then | 107.5 | 98.0 | 96.0 | 4.5% Zn |
| | | | cobalt nitrate, then | | | | |
| | | | tetraamminecopper | | | | |
| K19 | | inv. | Cobalt nitrate. then | 107.5 | 103.0 | 96.0 | 1.8% Co |
| | | | zinc nitrate. then | | | | |
| | | | tetraamminecopper | | | | |
| K20 | No final | comp. | 2x tetraamminecopper | 107.5 | 75.0 | n.a. | n.a. |
| | calcination | | dried under N2 | | | | |
| K21 | | inv. | Zinc nitrate, | 107.5 | 90.0 | 74.0 | 16.1% Zn |
| | | | calcined, then 2x | | | | |
| | | | tetraamminecopper, | | | | |
| | | | dried under N2 | | | | |

| | | Calci- | | | Calci- | Copper | | |
|---|---|---|---|---|---|---|---|---|
| | | nation | | | nation | content | ph of | |
| | | after | | | after | as | copper | |
| | | 1st | | | 2nd | Cu/% | salt | Final |
| Ex. | Metal | impreg- | Dopant | Metal | impreg- | by | solu- | calci- |
| no. | salt 1 | nation | metal 2 | salt 2 | nation | mass | tion | nation |
| K1 | n.a. | n.a. | n.a. | n.a. | n.a. | 12.4 | 10 | 450° C. |
| K2 | Zn(NO3)2•4H2O | 450° C. | n.a. | n.a. | n.a. | 8.6 | 10 | 450° C. |
| K3 | Zn(NO3)2•4H2O | 450° C. | n.a. | n.a. | n.a. | 10.7 | 10 | 450° C. |
| K4 | Zn(NH3)4CO3 | n.a. | n.a. | n.a. | n.a. | 8.5 | 10 | 450° C. |
| | solution | | | | | | | |
| K5 | Fe(NO3)3•9H2O | 450° C. | n.a. | n.a. | n.a. | 11.1 | 10 | 450° C. |
| K6 | Co(NO3)2•6H2O | 450° C. | n.a. | n.a. | n.a. | 11.4 | 10 | 450° C. |
| K7 | Mn(NO3)2•4H2O | 450° C. | n.a. | n.a. | n.a. | 11.3 | 10 | 450° C. |
| K8 | Mg(NO3)2•6H2O | 450° C. | n.a. | n.a. | n.a. | 11.1 | 10 | 450° C. |
| K9 | Voxelate solution | 350° C. | n.a. | n.a. | n.a. | 11.0 | 10 | 450° C. |
| K10 | n.a. | n.a. | n.a. | n.a. | n.a. | 23.3 | acidic | 450° C. |
| K11 | n.a. | n.a. | n.a. | n.a. | n.a. | 21.6 | 10 | 450° C. |
| K12 | Zn(NO3)2•4H2O | 450° C. | n.a. | n.a. | n.a. | 17.2 | 10 | 450° C. |
| K13 | Zn(NO3)2•4H2O | 450° C. | n.a. | n.a. | n.a. | 25.8 | acidic | 450° C. |
| K14 | Fe(NO3)3•9H2O | 450° C. | n.a. | n.a. | n.a. | 19.6 | 10 | 450° C. |
| K15 | Fe(NO3)3•9H2O | 450° C. | 4.5% Zn | Zn(NO3)2 | 450° C. | 10.4 | 10 | 450° C. |
| | | | | solution | | | | |
| K16 | Zn(NO3)2 | 450° C. | 1.8% Fe | Fe(NO3)3•9H2O | 450° C. | 10.4 | 10 | 450° C. |
| | solution | | | | | | | |
| K17 | Fe(NO3)3•9H2O | 450° C. | 4.5% Zn | Zn(NO3)2 | n.a. | 10.4 | 10 | 450° C. |
| | | | | solution | | | | |
| K18 | Zn(NO3)2 | 450° C. | 1.8% Co | Co(NO3)2•6H2O | 450° C. | 10.4 | 10 | 450° C. |
| | solution | | | | | | | |
| K19 | Co(NO3)2•6H2O | 450° C. | 4.5% Zn | Zn(NO3)2 | 450° C. | 10.4 | 10 | 450° C. |
| | | | | solution | | | | |
| K20 | n.a. | n.a. | n.a. | n.a. | n.a. | 18.0 | 10 | no |
| K21 | Zn(NO3)2 | 450° C. | n.a. | n.a. | n.a. | 15.2 | 10 | no |
| | solution | | | | | | | |

The metal contents are based on the hydrogenation catalyst in the reduced state (after reduction with hydrogen). If impregnation was effected only once or twice, the final calcination is identical to the calcination after the first (or second) impregnation. n. a.=not applicable; inv.=inventive.

Hydrogenation Experiments

The catalysts produced as described above were used in the hydrogenation of nitrobenzene to aniline. For this purpose, the respective catalysts were transferred into a fixed bed reactor in the oxidized state, and nitrogen was passed through it until the remaining oxygen had been driven out. The temperature was adjusted to values within a range from 200° C. to 240° C., and the activation was commenced by metering in hydrogen. The exothermicity caused by the reaction should be kept as low as possible.

For the reaction, nitrobenzene (NB) was metered into the activated catalyst, successively increasing and adjusting the amount of nitrobenzene to the target load of 0.9 g_{NB} ml_{cat}⁻¹h⁻¹. The molar hydrogen:nitrobenzene ratio was 10:1. The reaction was conducted polytropically, with removal of the heat formed in the reaction by a heat carrier. The hydrogenation was conducted in each case until breakthrough of nitrobenzene and hence incomplete conversion was observed. On conclusion of the reaction, nitrogen was passed through the catalyst to remove the excess hydrogen. Air at 260° C. to 320° C. was passed through the deactivated catalyst for reactivation, until the resulting exothermicity had abated. As a result, the catalyst was in the oxidic state again, and then it was possible to start a second run according to the same procedure.

The results with regard to the durations of the runs and aniline selectivities are recorded in table 2. The catalysts were used and assessed at least in two successive runs.

TABLE 2
Overview of the hydrogenation experiments conducted

| | Catalyst | Duration of | Selectivity | Duration of | Selectivity | Duration of | Selectivity |
|---|---|---|---|---|---|---|---|
| Ex. No. | from ex. | run 1/h | 1/h | run 2/h | 2/h | run 3/h | 3/h |
| H1 | K1 | 240 | 99.6 | 180 | 99.6 | n.a. | n.a. |
| H2 | K2 | 240 | 99.5 | 240 | 99.6 | n.a. | n.a. |
| H3 | K3 | 70 | 99.3 | 70 | 99.5 | n.a. | n.a. |
| H4 | K4 | 170 | 98.5 | 140 | 99.6 | n.a. | n.a. |
| H5 | K5 | 400 | 99.8 | 310 | 99.8 | n.a. | n.a. |
| H6 | K6 | 250 | 99 | 190 | 99 | n.a. | n.a. |
| H7 | K7 | 340 | 99.6 | 280 | 99.6 | n.a. | n.a. |
| H8 | K8 | 150 | 99.2 | 150 | 99.4 | n.a. | n.a. |
| H9 | K9 | 200 | 99.1 | 220 | 99.1 | n.a. | n.a. |
| H10 | K10 | 80 | 99.2 | 65 | 99.6 | n.a. | n.a. |
| H11 | K11 | 380 | 99.6 | 310 | 99.6 | n.a. | n.a. |
| H12 | K12 | 380 | 99.7 | 380 | 99.7 | 380 | 99.7 |
| H13 | K13 | 170 | 99.7 | 290 | 99.8 | n.a. | n.a. |
| H14 | K14 | 1070 | 99.8 | 950 | 99.9 | n.a. | n.a. |
| H15 | K15 | 580 | 99.8 | 500 | 99.9 | n.a. | n.a. |
| H16 | K16 | 300 | 99.8 | 250 | 99.7 | n.a. | n.a. |
| H17 | K17 | 320 | 99.7 | 280 | 99.8 | n.a. | n.a. |
| H18 | K18 | 240 | 99.2 | 210 | 99.3 | n.a. | n.a. |
| H19 | K19 | 210 | 99.2 | 180 | 99.2 | n.a. | n.a. |
| H20 | K20 | 770 | 99.8 | 480 | 99.7 | n.a. | n.a. |
| H21 | K21 | 400 | 99.7 | 370 | 99.7 | n.a. | n.a. |

As can be seen, the use of dopant elements has a significant influence on catalyst performance. In particular, the elements zinc and iron were found to be suitable additions in order to stabilize the duration of the second run or to increase the duration of the first run, while magnesium was found to be unsuitable. In example K11, it was possible to produce a catalyst having a long duration of run and selectivity that was stable over three hydrogenation experiments, as apparent from example H11. With the catalyst from example K14, it was even possible to combine the positive influence of two dopant elements (see the corresponding hydrogenation experiment H14).

1. A process for preparing a doped tetraamminecopper salt-based hydrogenation catalyst suitable for hydrogenation of an aromatic nitro compound to obtain an aromatic amine, said hydrogenation catalyst comprising copper in metallic or oxidic form and a dopant metal in metallic or oxidic form on a support, said support comprising shaped silicon dioxide bodies and/or shaped silicon carbide bodies, said process comprising:
(a) dissolving a metal salt comprising an iron salt, a cobalt salt, a manganese salt, a vanadium salt, a zinc salt, or a mixture of any two or more thereof in water or an aqueous ammonia solution to obtain an aqueous metal salt solution;
(b) treating the support with the aqueous metal salt solution to obtain a first impregnated catalyst precursor;
(c) drying the first impregnated catalyst precursor to obtain a first dried catalyst precursor;
(d) calcining the first dried catalyst precursor to obtain a first calcined catalyst precursor;
(e) dissolving a copper salt in aqueous ammonia to obtain an ammoniacal copper salt solution;
(f) treating the first calcined catalyst precursor with the ammoniacal copper salt solution to obtain a second impregnated catalyst precursor; and
(g) forming the doped tetraamminecopper salt-based hydrogenation catalyst by
(1) drying the second impregnated catalyst precursor
or
(2) drying and calcining the second impregnated catalyst precursor.

2. The process as claimed in claim 1,
in which, in step (b), for the treatment of 100 g of the support T, such a volume of aqueous metal salt solution V_{MS}(100 g T) is used that the ratio of the numerical value of the volume V_{MS}(100 g T) reported in milliliters to the numerical value of the maximum absorptivity of the support S_{T} to be treated, expressed in percent, is not more than 1.00:
[V_{MS}(100 g T)/ml]/[S_{T}/%]≤1.00
where the maximum absorptivity of the support S_{T} is calculated from the ratio of the maximum mass of demineralized water m_{H2O} that can be absorbed by a sample P_{T} of the support to the mass of the sample of the support m_{PT}, multiplied by 100%:
S_{T}=[m_{H2O}/m_{PT}]×100%;
and/or
in which in step (f), for the treatment of 100 g of the first calcined catalyst precursor KV1, such a volume of ammoniacal copper salt solution V_{KS}(100 g KV1) is used that the ratio of the numerical value of the volume V_{KS}(100 g KV1) expressed in milliliters to the numerical value of the maximum absorptivity of the first calcined catalyst precursor S_{KV1} to be treated, expressed in percent, is not more than 1.00:
[V_{KS}(100 g KV1)/ml]/[S_{KV1}/%]≤1.00
where the maximum absorptivity of the first calcined catalyst precursor S_{KV1} is calculated from the ratio of the maximum mass of demineralized water m_{H2O} that can be absorbed by a sample P_{KV1} of the first calcined catalyst precursor to the mass of the sample of the first calcined catalyst precursor m_{PKV1}, multiplied by 100%:
S_{KV1}=[m_{H2O}/m_{PK1}]×100%.

3. The process as claimed in claim 1,
in which the metal salt comprises a metal nitrate or metal oxalate;
and/or
in which the copper salt comprises copper hydroxide carbonate.

4. The process as claimed in claim 1, in which, in step (e), in addition to the copper salt, an ammonium salt is also dissolved in the aqueous ammonia.

5. The process as claimed in claim 1,
in which the treating in steps (b) and/or (f) comprises impregnating the support or the first catalyst precursor with the metal salt solution or the ammoniacal copper salt solution;
or
in which the treating in steps (b) and/or (f) comprises spraying the support or the first catalyst precursor with the metal salt solution or the ammoniacal copper salt solution.

6. The process as claimed in claim 1, in which the shaped silicon dioxide or silicon carbide bodies are (i) spheres, (ii) cylinders or (iii) aggregates of multiple cylinders joined to one another along their longitudinal axis and have an average diameter within a range from 1.0 mm to 15 mm, where the average diameter in the case of cylinders relates to the footprint of the cylinder, and in the case of aggregates composed of multiple cylinders joined to one another in their longitudinal direction to a circle that encloses the footprints of the mutually joined cylinders.

7. A doped tetraamminecopper salt-based hydrogenation catalyst obtained by the process of claim 1.

8. A process for preparing an aromatic amine by hydrogenating an aromatic nitro compound, comprising:
(I) providing a doped tetraamminecopper salt-based hydrogenation catalyst as claimed in claim 7;
(II) optionally activating the hydrogenation catalyst by treating with hydrogen in the absence of the aromatic nitro compound; and
(III) reacting the aromatic nitro compound with hydrogen in the presence of the optionally activated hydrogenation catalyst to obtain the aromatic amine.

9. The process as claimed in claim 8, in which the hydrogenation catalyst used is a tetraamminecopper carbonate-based hydrogenation catalyst.

10. The process as claimed in claim 9, in which the hydrogenation catalyst used is a tetraamminecopper carbonate ammonium salt-based hydrogenation catalyst.

11. The process as claimed in claim 8, in which step (I) comprises:
(a) dissolving a metal salt comprising an iron salt, a cobalt salt, a manganese salt, a vanadium salt, a zinc salt, or a mixture of any two or more thereof in water or aqueous ammonia solution to obtain an aqueous metal salt solution;
(b) treating the support with the aqueous metal salt solution to obtain a first impregnated catalyst precursor;
(c) drying the first impregnated catalyst precursor to obtain a first dried catalyst precursor;
(d) calcining the first dried catalyst precursor to obtain a first calcined catalyst precursor;
(e) dissolving a copper salt in aqueous ammonia to obtain an ammoniacal copper salt solution;
(f) treating the first calcined catalyst precursor with the ammoniacal copper salt solution to obtain a second impregnated catalyst precursor; and
(g) forming the doped tetraamminecopper salt-based hydrogenation catalyst by
(1) drying the second impregnated catalyst precursor
or
(2) drying and calcining the second impregnated catalyst precursor.

12. The process as claimed in claim 11, in which
in step (b), for the treatment of 100 g of the support T, such a volume of aqueous metal salt solution V_{MS}(100 g T) is used that the ratio of the numerical value of the volume V_{MS}(100 g T) expressed in milliliters to the numerical value of the maximum absorptivity of the support S_{T} to be treated, expressed in percent, is not more than 1.00:
[V_{MS}(100 g T)/ml]/[S_{T}/%]≤1.00
where the maximum absorptivity of the support S_{T} is calculated from the ratio of the maximum mass of demineralized water m_{H2O} that can be absorbed by a sample P_{T} of the support to the mass of the sample of the support m_{PT}, multiplied by 100%:
S_{T}=[m_{H2O}/m_{PT}]×100%;
and/or in which
in step (f), for the treatment of 100 g of the first calcined catalyst precursor KV1, such a volume of ammoniacal copper salt solution V_{KS}(100 g KV1) is used that the ratio of the numerical value of the volume V_{KS}(100 g T) expressed in milliliters to the numerical value of the maximum absorptivity of the first calcined catalyst precursor S_{KV1} to be treated, expressed in percent, is not more than 1.00:
[V_{KS}(100 g KV1)/ml]/[S_{KV1}/%]≤1.00
where the maximum absorptivity of the first calcined catalyst precursor S_{KV1} is calculated from the ratio of the maximum mass of demineralized water m_{H2O} that can be absorbed by a sample P_{KV1} of the first calcined catalyst precursor to the mass of the sample of the first calcined catalyst precursor m_{PKV1}, multiplied by 100%:
S_{KV1}=[m_{H2O}/m_{PKV1}]×100%.

13. The process as claimed in claim 11,
in which the treating in steps (b) and/or (f) comprises impregnating the support or the first catalyst precursor with the metal salt solution or the ammoniacal copper salt solution;
or in which
the treating in steps (b) and/or (f) comprises spraying the support or the first catalyst precursor with the metal salt solution or the ammoniacal copper salt solution.

14. The process as claimed in claim 8,
in which the metal salt comprises a metal nitrate or metal oxalate;
and/or
in which the copper salt comprises copper hydroxide carbonate;
and/or
in which in step (I)(e), in addition to the copper salt, an ammonium salt is also dissolved in the aqueous ammonia.

15. The process as claimed in claim 8, in which the optionally activated hydrogenation catalyst is arranged in a fixed catalyst bed in step (III).

16. The process as claimed in claim 1, wherein steps (a) to (c) or (a) to (d) are conducted repeatedly.

17. The process as claimed in claim 1, wherein steps (e) to (g)(1) or (e) to (g)(2) are conducted repeatedly.

18. The process as claimed in claim 12,
in which the treating in steps (b) and/or (f) comprises impregnating the support or the first catalyst precursor with the metal salt solution or the ammoniacal copper salt solution;
or in which
the treating in steps (b) and/or (f) comprises spraying the support or the first catalyst precursor with the metal salt solution or the ammoniacal copper salt solution.
